(19) United States
(12) Patent Application Publication
(10) Pub. No.: US 2025/0387313 A1
(43) Pub. Date: Dec. 25, 2025

(54) HYALURONIC ACID HYDROGEL WITH CERAMIDE BONDED THERETO

(71) Applicant: AMOREPACIFIC CORPORATION, Seoul (KR)

(72) Inventors: Jiwook JANG, Yongin-si (KR); Sungyeon CHO, Yongin-si (KR); Hyuk KIM, Yongin-si (KR); Byungryol PAIK, Yongin-si (KR); Jongsuk LEE, Yongin-si (KR)

(73) Assignee: AMOREPACIFIC CORPORATION, Seoul (KR)

(21) Appl. No.: 19/246,379

(22) Filed: Jun. 23, 2025

(30) Foreign Application Priority Data
Jun. 24, 2024 (KR) 10-2024-0082048

(51) Int. Cl. A61K 8/73; A61K 8/04; A61K 8/42

(52) U.S. Cl. CPC A61K 8/735; A61K 8/042; A61K 8/42

(57) ABSTRACT

One aspect of the present disclosure relates to a ceramide-bound hyaluronic acid hydrogel, and more particularly, the ceramide-bound hyaluronic acid hydrogel includes: a hyaluronic acid hydrogel including hyaluronic acid or a salt thereof; and emulsified particles bound to the hyaluronic acid hydrogel, wherein the emulsified particles includes at least one of a ceramide or a pseudoceramide. By simply mixing the ceramide-bound hyaluronic acid hydrogel according to one aspect of the present disclosure, a composition, particularly a composition of a solubilized formulation, including a ceramide and hyaluronic acid stably can be provided.

### CROSS REFERENCE TO RELATED APPLICATION

The present application claims priority to Korean Patent Application No. 10-2024-0082048, filed Jun. 24, 2024, the entire contents of which are hereby incorporated by this reference.

### BACKGROUND OF THE INVENTION

### Field of the Invention

The present disclosure relates to a ceramide-bound hyaluronic acid hydrogel.

### Description of the Related Art

Hyaluronic acid is known as a material with excellent moisturizing power and hydrophilic properties, and in particular, a cross-linked hyaluronic acid hydrogel has excellent moisture retention power. Ceramides are representative skin components that reinforce the barrier function of the skin, and human skin possesses various ceramides. Ceramides have a relatively small number of hydrophilic groups and thus exhibit strong hydrophobic interactions in the skin, and due to these structural features, it is possible to exhibit the inherent skin barrier functions of ceramides by preventing moisture loss from the human body and blocking the ingress of harmful ingredients from the external environment through the skin. Therefore, the application of ceramides to cosmetics increases the moisturizing power of the cosmetics and significantly enhances the skin-protecting power of the cosmetics, contributing to a significant improvement in the quality of the cosmetics.

Thus, various studies have been conducted to prepare a composition containing both of these ingredients capable of providing moisturizing power to the skin. In the related art, methods of i) dispersing a ceramide in hyaluronic acid using an emulsification process, or ii) chemically cross-linking a ceramide and hyaluronic acid have been usually used in order to prepare a composition including both a ceramide and hyaluronic acid. In the case of i) above, the final formulation is an emulsified formulation, which causes a white clouding phenomenon, and thus has problems in that the phenomenon is unsightly in appearance and it is difficult to apply the formulation as a solubilized formulation. In the case of an emulsified formulation, dispersed ceramide particles are likely to aggregate with each other. In the case of ii) above, a cross-linking agent is required in a mole equal to or greater than that of the ceramide for chemical bonding, and there are disadvantages in that an organic solvent-based reaction process and a purification process are indispensably required.

### SUMMARY OF THE INVENTION

An object of one aspect of the present disclosure is to provide a ceramide-bound hyaluronic acid hydrogel.

An object of one aspect of the present disclosure is to provide a composition including the ceramide-bound hyaluronic acid hydrogel.

An object of one aspect of the present disclosure is to provide a method for preparing a ceramide-bound hyaluronic acid hydrogel.

An object of one aspect of the present disclosure is to provide a method for preparing a composition including the ceramide-bound hyaluronic acid hydrogel.

The ceramide-bound hydrogel composition according to one aspect of the present disclosure includes:
a hyaluronic acid hydrogel including hyaluronic acid or a salt thereof; and emulsified particles bound to the hyaluronic acid hydrogel;
wherein the emulsified particles include at least one of a ceramide or a pseudoceramide.

The composition according to one aspect of the present disclosure includes a ceramide-bound hyaluronic acid hydrogel according to one aspect of the present disclosure.

A method for preparing the ceramide-bound hyaluronic acid hydrogel according to one aspect of the present disclosure includes: (a) mixing at least one of a ceramide or a pseudoceramide, an emulsifier and water to prepare a ceramide emulsified composition; and (b) mixing the prepared ceramide emulsified composition with at least one of hyaluronic acid or a salt thereof, and a cross-linking agent to obtain a hyaluronic acid hydrogel to which ceramide emulsified particles including at least one of a ceramide or a pseudoceramide are bound.

The method for preparing the composition according to one aspect of the present disclosure includes: mixing the ceramide-bound hyaluronic acid hydrogel according to one aspect of the present disclosure with purified water.

When the ceramide-bound hyaluronic acid hydrogel according to one aspect of the present disclosure is used, a composition stably including a ceramide (or a pseudoceramide) and hyaluronic acid (or a salt thereof) can be provided, and in particular, a composition of a solubilized formulation can be provided.

The ceramide-bound hyaluronic acid hydrogel according to one aspect of the present disclosure can be simply added to an oil-and emulsifier-free water-based formulation to provide a composition stably including both a ceramide and a hyaluronic acid gel.

A composition including the ceramide-bound hyaluronic acid hydrogel according to one aspect of the present disclosure can exhibit a transparent appearance, thereby increasing consumer satisfaction in terms of appearance. Meanwhile, a composition of an emulsified formulation obtained by emulsifying a ceramide in hyaluronic acid is not transparent, but has a white clouded appearance.

A composition including a ceramide-bound hydrogel composition according to one aspect of the present disclosure can exhibit excellent moisturizing power and usability.

### BRIEF DESCRIPTION OF THE DRAWINGS

FIG. 1 is a schematic view exemplarily illustrating a form in which a ceramide is bound to a hyaluronic acid hydrogel according to one aspect of the present disclosure.

FIG. 2 is a microscopy image of a ceramide-bound hyaluronic acid hydrogel according to an example.

FIG. 3 illustrates a set of confocal microscopy images of a ceramide-bound hyaluronic acid hydrogel according to an example.

FIG. 4 is a photograph comparing the appearance of a composition including a ceramide-bound hyaluronic acid hydrogel according to an example with the appearance of a composition in which hyaluronic acid and a ceramide are emulsified.

FIG. 5 is a graph comparing the moisturizing power of a composition including a ceramide-bound hyaluronic acid hydrogel according to an example with the moisturizing power of a composition in which hyaluronic acid and a ceramide are emulsified.

### DETAILED DESCRIPTION OF THE INVENTION

It should be understood that various exemplary embodiments of the present document and the terms used therein are not intended to limit the technical features set forth herein to particular exemplary embodiments and include various changes, equivalents, or replacements for a corresponding exemplary embodiment.

The present inventors have developed a hyaluronic acid hydrogel including a high content of ceramide by binding emulsified particles including the ceramide to a hyaluronic acid hydrogel, and have confirmed that when a ceramide-bound hyaluronic acid hydrogel is used, a composition stably including both a ceramide (or a pseudoceramide) and hyaluronic acid (or a salt thereof) can be prepared through simple mixing without an emulsification process.

### Ceramide-Bound Hyaluronic Acid Hydrogel

One aspect of the present disclosure provides a ceramide-bound hyaluronic acid hydrogel including: a hyaluronic acid hydrogel including hyaluronic acid or a salt thereof; and emulsified particles bound to the hyaluronic acid hydrogel, wherein the emulsified particles include at least one of a ceramide or a pseudoceramide.

In the present disclosure, the term “hydrogel” may refer to a structure formed by cross-linking hydrophilic polymers by covalent or non-covalent bonds. In this case, the structure may be a three-dimensional network structure. Hydrogels can absorb a large amount of water and swell under an aqueous environment in an aqueous solution due to the hydrophilicity of their constituent materials, but they may not be dissolved by their cross-linked structure.

The ceramide-bound hyaluronic acid hydrogel according to one aspect of the present disclosure may be obtained by physically and/or chemically stabilizing a ceramide, which is known to be poorly soluble. In an embodiment, the ceramide-bound hyaluronic acid hydrogel according to one aspect of the present disclosure may be obtained by binding a ceramide to a hydrogel including hyaluronic acid or a salt thereof to physically and/or chemically stabilize the ceramide. In an example, the ceramide or pseudoceramide was uniformly distributed in the hyaluronic acid hydrogel according to one aspect of the present disclosure.

In an embodiment, the emulsified particles including at least one of a ceramide or a pseudoceramide may be bound to the hydrogel including the hyaluronic acid or a salt thereof.

In an embodiment, the bond may include at least one of a physical bond or a chemical bond, but is not limited thereto. In an embodiment, the bond may be at least one of a physical bond or a chemical bond, but is not limited thereto. As used herein, “bond” may also be expressed as “bind”, “bound” or “binding”.

In an embodiment, a first component being bound to a second component may mean that the first component is disposed or located on at least a portion of the second component; or that the first component is disposed or located on at least a portion of the interior of and/or on the surface of the second component; or that the first component is disposed or located in contact with at least a portion of the second component; or that the first component is disposed or located in contact with at least a portion of the interior of and/or on the surface of the second component.

In an embodiment, the emulsified particles bound to the hyaluronic acid hydrogel may be emulsified particles disposed or located on at least a portion of the hyaluronic acid hydrogel; or emulsified particles disposed or located on at least a portion of the interior of and/or on the surface of the hyaluronic acid hydrogel. In an embodiment, the emulsified particles bound to the hyaluronic acid hydrogel may be emulsified particles disposed or located in contact with at least a portion of the hyaluronic acid hydrogel; or emulsified particles disposed or located in contact with at least a portion of the interior of and/or on the surface of the hyaluronic acid hydrogel.

In an embodiment, the ceramide-bound hyaluronic acid hydrogel may be one in which the ceramide is located or disposed in at least a portion of the hyaluronic acid hydrogel; or one in which the ceramide is located or disposed on at least a portion of the interior of and/or on the surface of the hyaluronic acid hydrogel. In this case, the hyaluronic acid hydrogel may further include other components in addition to the ceramide without any limitation.

In an embodiment, the ceramide-bound hyaluronic acid hydrogel may be one in which the ceramide is located or disposed in contact with at least a portion of the hyaluronic acid hydrogel, or one in which the ceramide is located or disposed in contact with at least a portion of the interior of and/or on the surface of the hyaluronic acid hydrogel. In this case, the hyaluronic acid hydrogel may further include other components in addition to the ceramide without any limitation.

In an embodiment, the binding may be encapsulating, but is not limited thereto.

In the present disclosure, the term “encapsulating” may refer to collecting a particular component, and the term “a first component is encapsulated in a second component” may mean that the first component is collected in the second component. For example, when a first component (for example: emulsified particles according to one aspect of the present disclosure) is encapsulated in a second component (for example: a hydrogel including hyaluronic acid or a salt thereof according to one aspect of the present disclosure), the first component may be disposed or located on at least a portion of the interior and/or on the surface of the second component, or the first component may be disposed or located in contact with at least a portion of the interior and/or on the surface of the second component. The encapsulation of the first component by the second component is not limited to the first component being surrounded by the second component.

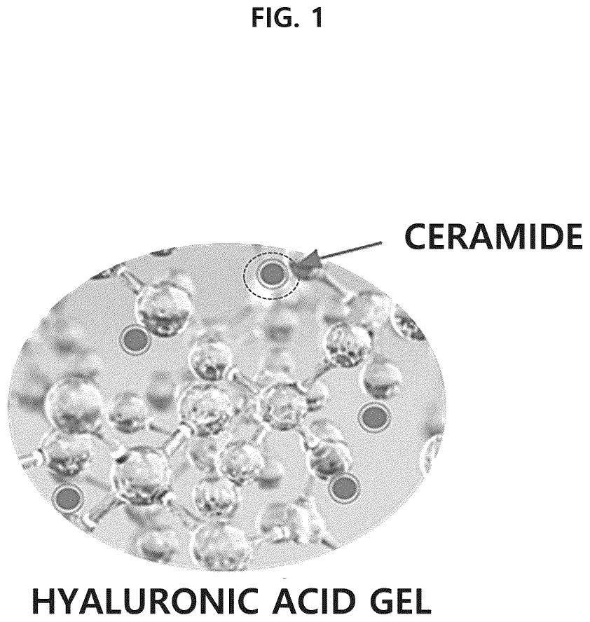

FIG. 1 is a schematic view exemplarily illustrating a form in which a ceramide is bound to a hyaluronic acid hydrogel according to one aspect.

In an embodiment, the ceramide-bound hyaluronic acid hydrogel may include hyaluronic acid or a salt thereof in an amount of 0.1 wt % to 10 wt % of based on the total weight of the ceramide-bound hyaluronic acid hydrogel, but the amount is not limited thereto. For example, the ceramide-bound hyaluronic acid hydrogel may have the hyaluronic acid or a salt thereof in an amount ranging from 0.1 wt % or more, 0.5 wt % or more, 1 wt % or more, 1.5 wt % or more, 2 wt % or more, 2.5 wt % or more, 3 wt % or more, 3.5 wt % or more, 4 wt % or more, 4.5 wt % or more, 5 wt % or more, 5.5 wt % or more, 6 wt % or more, 6.5 wt % or more, 7 wt % or more, 7.5 wt % or more, 8 wt % or more, 8.5 wt % or more, 9 wt % or more, 9.5 wt % or more, 10 wt % or more, 0.1 wt % or less, 0.5 wt % or less, 1 wt % or less, 1.5 wt % or less, 2 wt % or less, 2.5 wt % or less, 3 wt % or less, 3.5 wt % or less, 4 wt % or less, 4.5 wt % or less, 5 wt % or less, 5.5 wt % or less, 6 wt % or less, 6.5 wt % or less, 7 wt % or less, 7.5 wt % or less, 8 wt % or less, 8.5 wt % or less, 9 wt % or less, 9.5 wt % or less, 10 wt % or less, or a combination thereof based on the total weight of the ceramide-bound hyaluronic acid hydrogel, but the amount is not limited thereto.

In an embodiment, the ceramide-bound hyaluronic acid hydrogel may contain at least one of a ceramide or a pseudoceramide in an amount of 0.01 wt % to 10 wt % based on the total weight of the ceramide-bound hyaluronic acid hydrogel, but the amount is not limited thereto. For example, the ceramide-bound hyaluronic acid hydrogel may include at least one of a ceramide or a pseudoceramide in an amount ranging from 0.01 wt % or more, 0.05 wt % or more, 0.1 wt % or more, 0.15 wt % or more, 0.18 wt % or more, 0.19 wt % or more, 0.2 wt % or more, 0.22 wt % or more, 0.23 wt % or more, 0.3 wt % or more, 0.4 wt % or more, 0.5 wt % or more, 1 wt % or more, 1.5 wt % or more, 2 wt % or more, 2.5 wt % or more, 3 wt % or more, 3.5 wt % or more, 4 wt % or more, 4.5 wt % or more, 5 wt % or more, 5.5 wt % or more, 6 wt % or more, 6.5 wt % or more, 7 wt % or more, 7.5 wt % or more, 8 wt % or more, 8.5 wt % or more, 9 wt % or more, 9.5 wt % or more, 10 wt % or more, 0.01 wt % or less, 0.05 wt % or less, 0.1 wt % or less, 0.15 wt % or less, 0.18 wt % or less, 0.19 wt % or less, 0.2 wt % or less, 0.22 wt % or less, 0.23 wt % or less, 0.3 wt % or less, 0.4 wt % or less, 0.5 wt % or less, 1 wt % or less, 1.5 wt % or less, 2 wt % or less, 2.5 wt % or less, 3 wt % or less, 3.5 wt % or less, 4 wt % or less, 4.5 wt % or less, 5 wt % or less, 5.5 wt % or less, 6 wt % or less, 6.5 wt % or less, 7 wt % or less, 7.5 wt % or less, 8 wt % or less, 8.5 wt % or less, 9 wt % or less, 9.5 wt % or less, 10 wt % or less, or a combination thereof (for example: 0.15 wt % to 0.25 wt %, or 1 wt % to 8 wt %) based on the total weight of the ceramide-bound hyaluronic acid hydrogel, but the amount is not limited thereto.

In an embodiment, the hyaluronic acid hydrogel including the hyaluronic acid or a salt thereof may be one in which hyaluronic acid or a salt thereof is chemically cross-linked by a cross-linking agent, but is not limited thereto. For example, a hyaluronic acid hydrogel including hyaluronic acid may be one in which the hyaluronic acid is chemically cross-linked by a cross-linking agent. For example, a hyaluronic acid hydrogel including a salt of hyaluronic acid may be one in which the hyaluronic acid is chemically cross-linked by a cross-linking agent, or one in which the salt of hyaluronic acid is chemically cross-linked by a cross-linking agent.

In an embodiment, the cross-linking agent is sufficient as long as it can cross-link hyaluronic acid and/or a salt thereof, and a specific type thereof is not limited.

In an embodiment, the cross-linking agent may be at least one selected from the group consisting of a sulfone-based cross-linking agent, an epoxide-based cross-linking agent, and a phosphate-based cross-linking agent, but is not limited thereto.

In an embodiment, the cross-linking agent may be at least one selected from the group consisting of polyethylene glycol diglycidyl ether, sodium trimetaphosphate, epichlorohydrin, epibromohydrin, 1,4-butandiol diglycidyl ether, ethylene glycol diglycidyl ether, 1,6-hexanediol diglycidyl ether, propylene glycol diglycidyl ether, polypropylene glycol diglycidyl ether, polytetramethylene glycol diglycidyl ether, neopentyl glycol diglycidyl ether, polyglycerol polyglycidyl ether, diglycerol polyglycidyl ether, glycerol polyglycidyl ether, trimethylpropane polyglycidyl ether, 1,2-(bis(2,3-epoxypropoxy) ethylene), pentaerythritol polyglycidyl ether, and sorbitol polyglycidyl ether, but is not limited thereto.

In an embodiment, the ceramide may be a natural ceramide. In an embodiment, the ceramide may be a ceramide precursor.

In an embodiment, the ceramide may be at least one selected from the group consisting of ceramide NP, ceramide NS, ceramide AS, ceramide EOS, ceramide NDS, ceramide ADS, ceramide EODS, ceramide AP, ceramide EOP, ceramide NH, ceramide AH, ceramide EOH, and hydroxypalmitoyl sphinganine, but is not limited thereto.

In an embodiment, the pseudoceramide may be a synthetic material having a function similar to ceramide (for example, skin protective action, moisture retention ability, and the like).

In an embodiment, the pseudoceramide is sufficient as long as it is a material having a function similar to ceramides, and a specific type thereof is not limited. In an embodiment, the pseudoceramide may be a commercially available pseudoceramide, and a specific type thereof is not limited.

In an embodiment, the pseudoceramide may be at least one selected from the group consisting of hydroxypropyl bispalmitamide MEA, hydroxypropyl bisstearamide MEA, hydroxypropyl bislauramide MEA, hydroxypropyl bis-isostearamide MEA, bis-hydroxyethyl tocopherylsuccinoylamido hydroxypropane, cetyl-PG hydroxyethyl palmitamide, myristoyl/palmitoyl oxostearamide/arachamide MEA, and bis-palmitoyl tromethamine, but is not limited thereto.

In an embodiment, the pseudoceramide may be at least one selected from the group consisting of compounds represented by the following Chemical Formulae 1 to 6, but is not limited thereto.

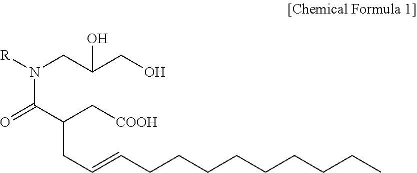

(in Chemical Formula 1, R is a C9-C21 saturated or unsaturated aliphatic chain.),

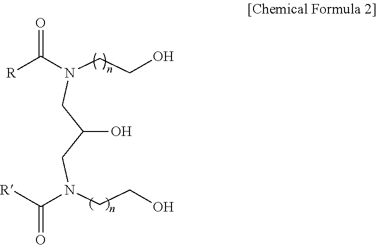

(in Chemical Formula 2, n is 1 or 2; R and R′ are each independently a C9-C21 saturated or unsaturated aliphatic chain.),

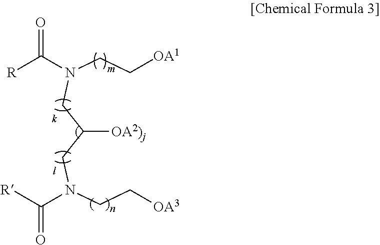

### (in Chemical Formula 3,

m and n are the same as or different from each other, and are an integer from 1 to 3;
k and I are the same as or different from each other, and are 1 or 2;
j is 0 or 1;
R and R′ are the same as or different from each other, and are a C1-C31 straight-chained or branched, saturated or unsaturated alkyl group with or without a hydroxyl group;
A¹, A² and A³ are the same as or different from each other, and are hydrogen or any one of the substituents having the following structures, provided that the case where A¹, A², and A³ are simultaneously hydrogen is excluded:

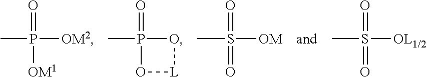

[in the formula, M, M¹ and M² are selected from the group consisting of alkali metals, lysine, arginine, histidine, triethanolamine, ammonia, polyquaternium-4, polyquaternium-6,polyquaternium-7, polyquaternium-10, polyquaternium-11, polyquaternium-16, lauryl dimethyl benzyl ammonium chloride and stearyl dimethyl benzyl ammonium chloride, and L is an alkaline earth metal.]),

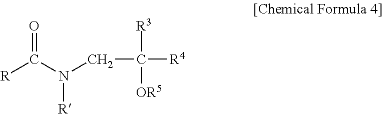

### (in Chemical Formula 4,

R and R′ are the same as or different from each other, and are a C10-C32 straight-chained or branched, saturated or unsaturated alkyl group with or without a hydroxyl group;
R³ and R⁴ are the same as or different from each other, and are hydrogen or a C1-C4 alkyl group or hydroxyalkyl group;
R⁵ is —A or —CH₂CH₂OA, where A is any one of the substituents having the following structures:

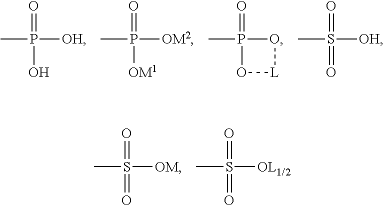

[in the formula, M, M¹ and M² are selected from the group consisting of alkali metals, lysine, arginine, histidine, triethanolamine, ammonia, polyquaternium-4, polyquaternium-6,polyquaternium-7, polyquaternium-10, polyquaternium-11, polyquaternium-16, lauryl dimethyl benzyl ammonium chloride and stearyl dimethyl benzyl ammonium chloride, and L is an alkaline earth metal.])

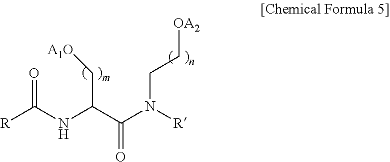

### (in Chemical Formula 5,

m and n are the same as or different from each other, and are an integer from 1 to 4;
R and R′ are the same as or different from each other, and are a C1-C31 straight-chained or branched, saturated or unsaturated alkyl group with or without a hydroxyl group;
A₁ and A₂ are the same as or different from each other, and are hydrogen or any one of the substituents having the following structures:

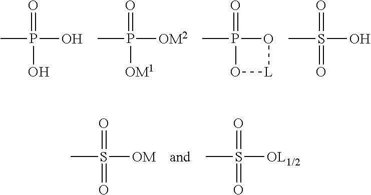

[in the formula, M, M¹ and M² are selected from the group consisting of alkali metals, lysine, arginine, histidine, triethanolamine, ammonia, polyquaternium-4, polyquaternium-6,polyquaternium-7, polyquaternium-10, polyquaternium-11, polyquaternium-16, lauryl dimethyl benzyl ammonium chloride and stearyl dimethyl benzyl ammonium chloride, and L is an alkaline earth metal.]),

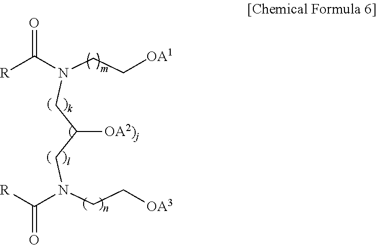

### (in Chemical Formula 6,

m and n are the same as or different from each other, and are an integer from 1 to 3;
k and 1 are the same as or different from each other, and are 1 or 2;
j is 0 or 1;
A¹, A² and A³ are the same as or different from each other, and are hydrogen or any one of the substituents having the following structures:

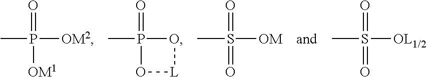

[in the formula, M, M¹ and M² are alkali metals or nitrogen-containing organic bases, and L is an alkaline earth metal.];
R is a substituent having the following structure:

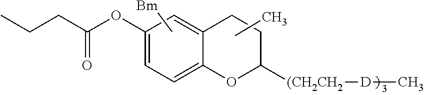

[in the formula, B is a methyl group at the 5-, 7-, or 8-position of tocopherol, m is an integer from 1 to 3, and D is —CH₂(CH₃)CH—or —CH(CH₃)═C—.])

A ceramide and/or a pseudoceramide are/is included in a hyaluronic acid hydrogel, and thus may exhibit the effect of strengthening the skin barrier and/or improving moisturizing power.

In an embodiment, hyaluronic acid or a salt thereof may have a molecular weight of 10,000 Da to 5,000,000 Da, but the molecular weight is not limited thereto. For example, hyaluronic acid or a salt thereof may have a molecular weight ranging from 10,000 Da or more, 20,000 Da or more, 30,000 Da or more, 40,000 Da or more, 50,000 Da or more, 60,000 Da or more, 70,000 Da or more, 80,000 Da or more, 90,000 Da or more, 100,000 Da or more, 200,000 Da or more, 300,000 Da or more, 400,000 Da or more, 500,000 Da or more, 600,000 Da or more, 700,000 Da or more, 800,000 Da or more, 900,000 Da or more, 1,000,000 Da or more, 1,500,000 Da or more, 2,000,000 Da or more, 2,500,000 Da or more, 3,000,000 Da or more, 3,500,000 Da or more, 4,000,000 Da or more, 4,500,000 Da or more, 5,000,000 Da or more, 10,000 Da or less, 20,000 Da or less, 30,000 Da or less, 40,000 Da or less, 50,000 Da or less, 60,000 Da or less, 70,000 Da or less, 80,000 Da or less, 90,000 Da or less, 100,000 Da or less, 200,000 Da or less, 300,000 Da or less, 400,000 Da or less, 500,000 Da or less, 600,000 Da or less, 700,000 Da or less, 800,000 Da or less, 900,000 Da or less, 1,000,000 Da or less, 1,500,000 Da or less, 2,000,000 Da or less, 2,500,000 Da or less, 3,000,000 Da or less, 3,500,000 Da or less, 4,000,000 Da or less, 4,500,000 Da or less, 5,000,000 Da or less, or a combination thereof (for example: 100,000 Da to 3,000,000 Da, or 900,000 Da to 1,100,000 Da), but the molecular weight is not limited thereto.

In an embodiment, the hyaluronic acid may be hyaluronic acid, but is not limited thereto.

In an embodiment, the salt of hyaluronic acid may be sodium hyaluronate, but is not limited thereto.

In an embodiment, the hyaluronic acid hydrogel including hyaluronic acid or a salt thereof may be a sodium hyaluronate crosspolymer, but is not limited thereto. The sodium hyaluronate crosspolymer may be a hydrogel obtained by cross-linking hyaluronic acid or a salt thereof.

In an embodiment, the emulsified particles may have an average diameter of, but not limited to, 0.1 μm to 100 μm. For example, the emulsified particles may have an average diameter ranging from 0.1 μm or more, 0.2 μm or more, 0.23 μm or more, 0.3 μm or more, 0.4 μm or more, 0.5 μm or more, 0.6 μm or more, 0.7 μm or more, 0.8 μm or more, 0.9 μm or more, 1 μm or more, 2 μm or more, 3 μm or more, 4 μm or more, 5 μm or more, 5.5 μm or more, 6μm or more, 7 μm or more, 8 μm or more, 9 μm or more, 10 μm or more, 20 μm or more, 30 μm or more, 40 μm or more, 50 μm or more, 60 μm or more, 70 μm or more, 80 μm or more, 90 μm or more, 100 μm or more, 0.1 μm or less, 0.2 μm or less, 0.23 μm or less, 0.3 μm or less, 0.4 μm or less, 0.5 μm or less, 0.6 μm or less, 0.7 μm or less, 0.8 μm or less, 0.9 μm or less, 1 μm or less, 2 μm or less, 3 μm or less, 4 μm or less, 5 μm or less, 5.5 μm or less, 6 μm or less, 7 μm or less, 8 μm or less, 9 μm or less, 10 μm or less, 20 μm or less, 30 μm or less, 40 μm or less, 50 μm or less, 60 μm or less, 70 μm or less, 80 μm or less, 90 μm or less, 100 μm or less, or a combination thereof (for example: 0.2 μm to 0.6 μm, or 0.9 μm to 5.5 μm), but the average diameter is not limited thereto.

In an embodiment, the ceramide-bound hyaluronic acid hydrogel according to one aspect of the present disclosure may be a pulverized ceramide-bound hyaluronic acid hydrogel. In an embodiment, the ceramide-bound hyaluronic acid hydrogel according to one aspect of the present disclosure may be a ceramide-bound hyaluronic acid hydrogel that is pulverized to exhibit the form of particles.

In an embodiment, the ceramide-bound hyaluronic acid hydrogel according to one aspect of the present disclosure may be in the form of particles. In an embodiment, the ceramide-bound hyaluronic acid hydrogel according to one aspect of the present disclosure may have a diameter of, but not limited to, 10 μm to 100 μm. For example, the ceramide-bound hyaluronic acid hydrogel according to one aspect of the present disclosure may have a diameter ranging from 10 μm or more, 15 μm or more, 20 μm or more, 25 μm or more, 30 μm or more, 35 μm or more, 40 μm or more, 45 μm or more, 50 μm or more, 55 μm or more, 60μm or more, 65 μm or more, 70 μm or more, 75 μm or more, 80 μm or more, 85 μm or more, 90 μm or more, 95 μm or more, 100 μm or more, 10 μm or less, 15 μm or less, 20 μm or less, 25 μm or less, 30 μm or less, 35 μm or less, 40 μm or less, 45 μm or less, 50 μm or less, 55 μm or less, 60 μm or less, 65 μm or less, 70 μm or less, 75 μm or less, 80 μm or less, 85 μm or less, 90 μm or less, 95 μm or less, 100 μm or less, or a combination thereof (for example: 40 μm to 60 μm), but the diameter is not limited thereto. In an embodiment, the ceramide-bound hyaluronic acid hydrogel according to one aspect of the present disclosure may have a diameter larger than the above range according to the pulverization process (for example: the ceramide-bound hyaluronic acid hydrogel according to one aspect of the present disclosure may have a diameter of, but not limited to, 0.01 cm or more, 0.1 cm or more, 1 cm or more, or 5 cm or more).

When the ceramide-bound hyaluronic acid hydrogel according to one aspect of the present disclosure includes a plurality of ceramide-bound hyaluronic acid hydrogel particles, the diameter may be an average diameter.

In an embodiment, the ceramide-bound hyaluronic acid hydrogel may further include other components (for example: oil, cholesterol, polyglyceryl, and the like) that may be typically included in the composition, if necessary.

In an embodiment, the Tand of the ceramide-bound hyaluronic acid hydrogel may be 0.01 to 2, but is not limited thereto. For example, the Tand of the ceramide-bound hyaluronic acid hydrogel may range from 0.01 or more, 0.02 or more, 0.03 or more, 0.04 or more, 0.05 or more, 0.06 or more, 0.07 or more, 0.08 or more, 0.09 or more, 0.1 or more, 0.2 or more, 0.3 or more, 0.4 or more, 0.5 or more, 0.6 or more, 0.7 or more, 0.8 or more, 0.9 or more, 1 or more, 1.05 or more, 1.08 or more, 1.09 or more, 1.1 or more, 1.2 or more, 1.3 or more, 1.4 or more, 1.5 or more, 1.6 or more, 1.7 or more, 1.8 or more, 1.9 or more, 2.0 or more, 0.01 or less, 0.02 or less, 0.03 or less, 0.04 or less, 0.05 or less, 0.06 or less, 0.07 or less, 0.08 or less, 0.09 or less, 0.1 or less, 0.2 or less, 0.3 or less, 0.4 or less, 0.5 or less, 0.6 or less, 0.7 or less, 0.8 or less, 0.9 or less, 1 or less, 1.05 or less, 1.08 or less, 1.09 or less, 1.1 or less, 1.2 or less, 1.3 or less, 1.4 or less, 1.5 or less, 1.6 or less, 1.7 or less, 1.8 or less, 1.9 or less, 2.0 or less, or a combination thereto (for example: 0.08 to 1.09), but is not limited thereto.

The Tanδ may be calculated by the following Mathematical Formula 1.

$$Tan δ = loss modulus measured at 25 ° C . ( Pa ) / storage modulus measured at 25 ° C . ( Pa ) . \qquad [ Mathematical Formula 1 ]$$

In an embodiment, the ceramide-bound hyaluronic acid hydrogel may have an elasticity of, but not limited to, 50 Pa to 1500 Pa at 25° C. In the present disclosure, the elasticity may also be expressed as storage modulus. For example, the ceramide-bound hyaluronic acid hydrogel may have an elasticity ranging from 50 Pa or more, 60 Pa or more, 70 Pa or more, 80 Pa or more, 90 Pa or more, 100 Pa or more, 110 Pa or more, 112 Pa or more, 120 Pa or more, 130 Pa or more, 140 Pa or more, 150 Pa or more, 160 Pa or more, 170 Pa or more, 180 Pa or more, 190 Pa or more, 200 Pa or more, 210 Pa or more, 220 Pa or more, 230 Pa or more, 240 Pa or more, 250 Pa or more, 260 Pa or more, 270 Pa or more, 280 Pa or more, 290 Pa or more, 300 Pa or more, 350 Pa or more, 400 Pa or more, 450 Pa or more, 500 Pa or more, 550 Pa or more, 600 Pa or more, 650 Pa or more, 700 Pa or more, 750 Pa or more, 760 Pa or more, 800 Pa or more, 850 Pa or more, 900 Pa or more, 950 Pa or more, 990 Pa or more, 1000 Pa or more, 1100 Pa or more, 1200 Pa or more, 1300 Pa or more, 1400 Pa or more, 1500 Pa or more, 50 Pa or less, 60 Pa or less, 70 Pa or less, 80 Pa or less, 90 Pa or less, 100 Pa or less, 110 Pa or less, 112 Pa or less, 120 Pa or less, 130 Pa or less, 140 Pa or less, 150 Pa or less, 160 Pa or less, 170 Pa or less, 180 Pa or less, 190 Pa or less, 200 Pa or less, 210 Pa or less, 220 Pa or less, 230 Pa or less, 240 Pa or less, 250 Pa or less, 260 Pa or less, 270 Pa or less, 280 Pa or less, 290 Pa or less, 300 Pa or less, 350 Pa or less, 400 Pa or less, 450 Pa or less, 500 Pa or less, 550 Pa or less, 600 Pa or less, 650 Pa or less, 700 Pa or less, 750 Pa or less, 760 Pa or less, 800 Pa or less, 850 Pa or less, 900 Pa or less, 950 Pa or less, 990 Pa or less, 1000 Pa or less, 1100 Pa or less, 1200 Pa or less, 1300 Pa or less, 1400 Pa or less, 1500 Pa or less, or a combination thereof (for example: 100 Pa to 760 Pa) at 25° C., but the elasticity is not limited thereto.

In an embodiment, the ceramide-bound hyaluronic acid hydrogel may have a viscosity of, but not limited to, 10 Pa to 500 Pa at 25° C. In the present disclosure, the viscosity may also be expressed as loss modulus. For example, the ceramide-bound hyaluronic acid hydrogel may have a viscosity ranging from 10 Pa or more, 20 Pa or more, 30 Pa or more, 40 Pa or more, 50 Pa or more, 60 Pa or more, 70 Pa or more, 80 Pa or more, 84 Pa or more, 90 Pa or more, 100 Pa or more, 110 Pa or more, 120 Pa or more, 121 Pa or more, 130 Pa or more, 140 Pa or more, 150 Pa or more, 160 Pa or more, 170 Pa or more, 180 Pa or more, 190 Pa or more, 200 Pa or more, 210 Pa or more, 220 Pa or more, 230 Pa or more, 240 Pa or more, 250 Pa or more, 260 Pa or more, 270 Pa or more, 274 Pa or more, 280 Pa or more, 300 Pa or more, 350 Pa or more, 400 Pa or more, 450 Pa or more, 500 Pa or more, 10 Pa or less, 20 Pa or less, 30 Pa or less, 40 Pa or less, 50 Pa or less, 60 Pa or less, 70 Pa or less, 80 Pa or less, 84 Pa or less, 90 Pa or less, 100 Pa or less, 110 Pa or less, 120 Pa or less, 121 Pa or less, 130 Pa or less, 140 Pa or less, 150 Pa or less, 160 Pa or less, 170 Pa or less, 180 Pa or less, 190 Pa or less, 200 Pa or less, 210 Pa or less, 220 Pa or less, 230 Pa or less, 240 Pa or less, 250 Pa or less, 260 Pa or less, 270 Pa or less, 274 Pa or less, 280 Pa or less, 300 Pa or less, 350 Pa or less, 400 Pa or less, 450 Pa or less, 500 Pa or less, or a combination thereof (for example: 84 Pa to 280 Pa) at 25° C., but the viscosity is not limited thereto.

In an embodiment, the ceramide-bound hyaluronic acid hydrogel may have a degree of swelling of, but not limited to, 10 to 300. For example, the ceramide-bound hyaluronic acid hydrogel may have a degree of swelling ranging from 10 or more, 20 or more, 30 or more, 40 or more, 50 or more, 60 or more, 70 or more, 80 or more, 90 or more, 100 or more, 110 or more, 120 or more, 130 or more, 140 or more, 150 or more, 160 or more, 170 or more, 180 or more, 190 or more, 200 or more, 210 or more, 220 or more, 230 or more, 240 or more, 250 or more, 260 or more, 270 or more, 280 or more, 290 or more, 300 or more, 10 or less, 20 or less, 30 or less, 40 or less, 50 or less, 60 or less, 70 or less, 80 or less, 90 or less, 100 or less, 110 or less, 120 or less, 130 or less, 140 or less, 150 or less, 160 or less, 170 or less, 180 or less, 190 or less, 200 or less, 210 or less, 220 or less, 230 or less, 240 or less, 250 or less, 260 or less, 270 or less, 280 or less, 290 or less, 300 or less, or a combination thereof (for example: 80 to 120), but the degree of swelling is not limited thereto.

In an embodiment, the degree of swelling may be measured as the weight of the hyaluronic acid hydrogel before drying compared to the weight of the hyaluronic acid hydrogel after drying.

In an embodiment, the degree of swelling may be measured by the following method for measuring a degree of swelling:

### Method for Measuring Degree of Swelling

Degree of swelling=weight of ceramide-bound hyaluronic acid hydrogel (i.e., weight of ceramide-bound hyaluronic acid hydrogel before drying)/weight of ceramide-bound hyaluronic acid hydrogel measured after drying at 60° C. for 12 hours.

The ceramide-bound hyaluronic acid hydrogel according to one aspect of the present disclosure may exhibit the effect of strengthening the skin barrier and/or improving moisturizing power.

### Composition Including Ceramide-Bound Hyaluronic Acid Hydrogel

One aspect of the present disclosure provides a composition including the above-described ceramide-bound hyaluronic acid hydrogel. Since the ceramide-bound hyaluronic acid hydrogel has been described above, the detailed description thereof will be omitted.

The composition according to one aspect of the present disclosure stably include a ceramide (or a pseudoceramide) in the composition without an emulsification process by including the above-described ceramide-bound hyaluronic acid hydrogel. In the related art, in order to stably include both a ceramide and hyaluronic acid in a composition, an emulsified formulation was prepared by an emulsification process.

The composition according to one aspect of the present disclosure may be prepared by simply mixing the above-described ceramide-bound hyaluronic acid hydrogel with water and/or any component that may be included in the composition.

In an embodiment, the composition may be a solubilized formulation.

In the present disclosure, the term “solubilization” may refer to a state in which a substance that is insoluble or partially soluble in water is dissolved transparently. The solubilized formulation is similar to an oil-in-water (O/W) formulation, but may be a formulation having a relatively high content of purified water (or water). Due to its relatively light weight, non-greasy properties, and the like, the solubilized formulation may be usually applied to a product such as a skin lotion, an emollient, a spray, a mist, and a perfume.

In an embodiment, the composition according to an aspect of the present disclosure may be transparent. This may enhance the consumers' satisfaction with the appearance. Meanwhile, an emulsified composition including both a ceramide and hyaluronic acid, which is prepared by an emulsification process in the related art is a white suspension formulation.

In an embodiment, the composition may be a composition for enhancing skin moisture. In an embodiment, the composition may be a composition for strengthening the skin barrier.

In an embodiment, the composition may be a cosmetic composition, a non-therapeutic composition, a pharmaceutical composition, a composition for external application, a food composition, an oral composition, or a non-therapeutic oral composition. For example, the composition may be a cosmetic composition, a pharmaceutical composition (for example: a pharmaceutical composition for transdermal administration), or a composition for external application (for example: a composition for external application to the skin). In an embodiment, the skin or the hair may be treated with the composition.

In an embodiment, the composition may further include other components typically blended in the composition, if necessary. For example, the composition may further include a moisturizer, an emollient, organic and inorganic pigments, an organic powder, an ultraviolet absorbent, a preservative, a bactericide, an antioxidant, a plant extract, a pH adjuster, an alcohol, a colorant, a fragrance, a circulation accelerator, a cooling agent, and/or purified water, and the like, but is not limited thereto. The amounts of the components blended can be varied without limitation within a range not impairing the objects and effects of the present disclosure.

In an embodiment, the composition may be formulated in the form of, but not limited to, a skin lotion, a toner, a spray, a mist, a perfume, a lotion, a cream, a pack, a gel, a powder, an ointment, or a concealer stick.

The composition according to one aspect of the present disclosure can be applied as various formulations. In particular, the composition according to one aspect of the present disclosure can be applied as a solubilized formulation, in which case it may be provided in the form of, but not limited to, a skin lotion, a toner, a spray, a mist, a cosmetic water, or a perfume.

### Method for Enhancing Skin Moisture or Strengthening Skin Barrier

One aspect of the present disclosure provides a method for improving the skin, the method including applying the above-described ceramide-bound hyaluronic acid hydrogel. One aspect of the present disclosure provides a method for improving the skin, the method including applying the above-described ceramide-bound hyaluronic acid hydrogel to a subject.

One aspect of the present disclosure provides a method for improving the skin, the method including applying a composition including the above-described ceramide-bound hyaluronic acid hydrogel. One aspect of the present disclosure provides a method for improving the skin, the method including applying a composition including the above-described ceramide-bound hyaluronic acid hydrogel to a subject.

Since the ceramide-bound hyaluronic acid hydrogel and the composition including the ceramide-bound hyaluronic acid hydrogel have been described above, the detailed description thereof will be omitted.

In an embodiment, the application may be oral administration, transdermal administration, subcutaneous administration, intravenous administration, intraperitoneal administration, intramuscular, embrocation or local embrocation. In an embodiment, the application may be application to the skin, but is not limited thereto.

In an embodiment, the subject may be an animal including a mammal (for example: a human), but is not limited thereto. In an embodiment, the subject may be an individual in need of skin moisturization or strengthened skin barrier. In an embodiment, the skin improvement may be enhanced skin moisturization or strengthened skin barrier.

In an embodiment, the method for improving the skin may be a method for enhancing skin moisture or strengthening the skin barrier, but is not limited thereto.

### Method for Preparing Ceramide-Bound Hyaluronic Acid Hydrogel

One aspect of the present disclosure provides a method for preparing a ceramide-bound hyaluronic acid hydrogel, the method including:
(a) mixing at least one of a ceramide or a pseudoceramide, an emulsifier, and water to prepare a ceramide emulsified composition; and
(b) mixing the prepared ceramide emulsified composition with at least one of hyaluronic acid or a salt thereof, and a cross-linking agent to obtain a hyaluronic acid hydrogel to which emulsified particles including at least one of a ceramide or a pseudoceramide are bound.

The method for preparing a ceramide-bound hyaluronic acid hydrogel according to one aspect of the present disclosure may be a method for preparing the above-described ceramide-bound hyaluronic acid hydrogel.

Since a ceramide, a pseudoceramide, hyaluronic acid, a salt thereof, a cross-linking agent, emulsified particles including at least one of the ceramide or the pseudoceramide, a ceramide-bound hyaluronic acid hydrogel, and a hydrogel have been described above, the detailed description thereof will be omitted.

In an embodiment, the emulsifier may be at least one selected from the group consisting of a polyglyceryl-based surfactant and an anionic surfactant, but is not limited thereto.

In an embodiment, the emulsifier may be at least one selected from the group consisting of polyglyceryl-3 methylglucose distearate, polyglyceryl-2 stearate, polyglyceryl-10 stearate, cetyl phosphate, sodium stearoyl glutamate, sodium stearoyl lactylate, glyceryl stearate citrate, and ceryl phosphate, but is not limited thereto.

In an embodiment, the water may be purified water and/or distilled water, but is not limited thereto.

In an embodiment, the ceramide emulsified composition may further include other components (for example: oil, cholesterol, polyglyceryl, and the like) that may be typically included in the composition, if necessary.

In an embodiment, the ceramide emulsified composition may include ceramide emulsified particles.

In an embodiment, the emulsified particles may have an average diameter of, but not limited to, 0.1 μm to 100 μm. For example, the emulsified particles may have an average diameter ranging from 0.1 μm or more, 0.2 μm or more, 0.23 μm or more, 0.3 μm or more, 0.4 μm or more, 0.5 μm or more, 0.6 μm or more, 0.7 μm or more, 0.8 μm or more, 0.9 μm or more, 1 μm or more, 2 μm or more, 3 μm or more, 4 μm or more, 5 μm or more, 5.5 μm or more, 6 μm or more, 7 μm or more, 8 μm or more, 9 μm or more, 10 μm or more, 20 μm or more, 30 μm or more, 40 μm or more, 50 μm or more, 60 μm or more, 70 μm or more, 80 μm or more, 90 μm or more, 100 μm or more, 0.1 μm or less, 0.2 μm or less, 0.23 μm or less, 0.3 μm or less, 0.4 μm or less, 0.5 μm or less, 0.6 μm or less, 0.7 μm or less, 0.8 μm or less, 0.9 μm or less, 1 μm or less, 2 μm or less, 3 μm or less, 4 μm or less, 5 μm or less, 5.5 μm or less, 6 μm or less, 7 μm or less, 8 μm or less, 9 μm or less, 10 μm or less, 20 μm or less, 30 μm or less, 40 μm or less, 50 μm or less, 60 μm or less, 70 μm or less, 80 μm or less, 90 μm or less, 100 μm or less, or a combination thereof (for example: 0.2 μm to 0.6 μm), but the average diameter is not limited thereto.

In an embodiment, the ceramide emulsified composition may be an oil-in-water (O/W) emulsified composition.

In an embodiment, the step (b) may be mixing and reacting the ceramide emulsified composition prepared in step (a) above with at least one of hyaluronic acid or a salt thereof, and a cross-linking agent to obtain a hyaluronic acid hydrogel to which emulsified particles including at least one of a ceramide or a pseudoceramide are bound. In this case, the reaction may be performed at 20° C. to 60° C., 25° C. to 60° C., 30° C. to 60° C., 30° C. to 50° C., 30° C. to 40° C., or 35° C. to 40° C., but the temperature is not limited thereto.

In an embodiment, the step (b) may be dispersing the ceramide emulsified composition prepared in step (a) above in an alkaline solution, and then mixing and reacting the resulting dispersion with at least one of hyaluronic acid or a salt thereof, and a cross-linking agent to obtain a hyaluronic acid hydrogel to which emulsified particles including at least one of a ceramide or a pseudoceramide are bound. In this case, the reaction may be carried out at 20° C. to 60° C., 25° C. to 60° C., 30° C. to 60° C., 30° C. to 50° C., 30°° C. to 40° C., or 35° C. to 40° C., but the temperature is not limited thereto.

In an embodiment, the alkaline solution may include sodium hydroxide, potassium hydroxide, tetramethylammonium hydroxide, ammonia, sodium carbonate, sodium bicarbonate, potassium carbonate, potassium bicarbonate, and other alkalis, but is not limited thereto.

In an embodiment, the step (b) the obtaining of the hyaluronic acid hydrogel to which emulsified particles including at least one of the ceramide or the pseudoceramide are bound may be mixing the ceramide emulsified composition prepared in step (a) above with hyaluronic acid or a salt thereof, and a cross-linking agent to obtain a gel mixture, and allowing the obtained gel mixture to be swollen and then pulverized to obtain a hyaluronic acid hydrogel to which emulsified particles including at least one of a ceramide or a pseudoceramide are bound.

In this case, in the step (b), the hyaluronic acid or the salt thereof may be included in an amount of more than 10 wt % to less than 30 wt % based on the total weight of the gel mixture, but the amount is not limited thereto. For example, the hyaluronic acid or a salt thereof may be included in an amount ranging from more than 10 wt %, 11 wt % or more, 12 wt % or more, 13 wt % or more, 14 wt % or more, 15 wt % or more, 16 wt % or more, 17 wt % or more, 18 wt % or more, 19 wt % or more, 20 wt % or more, 21 wt % or more, 22 wt % or more, 23 wt % or more, 24 wt % or more, 25 wt % or more, 26 wt % or more, 27 wt % or more, 28 wt % or more, 29 wt % or more, 11 wt % or less, 12 wt % or less, 13 wt % or less, 14 wt % or less, 15 wt % or less, 16 wt % or less, 17 wt % or less, 18 wt % or less, 19 wt % or less, 20 wt % or less, 21 wt % or less, 22 wt % or less, 23 wt % or less, 24 wt % or less, 25 wt % or less, 26 wt % or less, 27 wt % or less, 28 wt % or less, 29 wt % or less, less than 30 wt %, or a combination thereof (for example: 15 wt % or more and less than 30 wt %, or 15 wt % to 25 wt %) based on the total weight of the gel mixture, but the amount is not limited thereto.

In an embodiment, the step (b) may be performed at 20° C. to 60° C., 25° C. to 60° C., 30° C. to 60° C., 30° C. to 50° C., 30° C. to 40° C., or 35° C. to 40° C., but the temperature is not limited thereto.

In an embodiment, the hyaluronic acid hydrogel to which emulsified particles including at least one of the ceramide or the pseudoceramide are bound obtained in (b) above may be in the form of particles.

In an embodiment, the hyaluronic acid hydrogel to which emulsified particles including at least one of the ceramide or the pseudoceramide are bound obtained in (b) above may have a diameter of, but not limited to, 10 μm to 100 μm. For example, the hyaluronic acid hydrogel to which emulsified particles including at least one of the ceramide or the pseudoceramide are bound obtained in (b) above may have a diameter ranging from 10 μm or more, 15 μm or more, 20 μm or more, 25 μm or more, 30 μm or more, 35 μm or more, 40 μm or more, 45 μm or more, 50 μm or more, 55 μm or more, 60 μm or more, 65 μm or more, 70 μm or more, 75 μm or more, 80 μm or more, 85 μm or more, 90 μm or more, 95 μm or more, 100 μm or more, 10 μm or less, 15 μm or less, 20 μm or less, 25 μm or less, 30 μm or less, 35 μm or less, 40 μm or less, 45 μm or less, 50 μm or less, 55 μm or less, 60 μm or less, 65 μm or less, 70 μm or less, 75 μm or less, 80 μm or less, 85 μm or less, 90 μm or less, 95 μm or less, 100 μm or less, or a combination thereof (for example: 40 μm to 60 μm), the diameter is not limited thereto. In an embodiment, the hyaluronic acid hydrogel to which emulsified particles including at least one of the ceramide or the pseudoceramide are bound obtained in (b) above may have a diameter larger than the above range according to the pulverization process (for example: the hyaluronic acid hydrogel to which emulsified particles including at least one of the ceramide or the pseudoceramide are bound obtained in (b) above may have a diameter of, but not limited to, 0.01 cm or more, 0.1 cm or more, 1 cm or more, or 5 cm or more.).

When the hyaluronic acid hydrogel to which emulsified particles including at least one of the ceramide or the pseudoceramide are bound obtained in (b) above includes a plurality of hydrogel particles, the diameter may be the average diameter of the plurality of hydrogel particles.

In an embodiment, the pulverization may be performed by an extruder equipped with a sieve.

### Method for Preparing Composition Including Ceramide-Bound Hyaluronic Acid Hydrogel

One aspect of the present disclosure provides a method for preparing a composition, the method including: i) mixing the above-described ceramide-bound hyaluronic acid hydrogel, or ii) mixing the ceramide-bound hyaluronic acid hydrogel prepared by the above-described method for preparing a ceramide-bound hyaluronic acid hydrogel with purified water.

Since the ceramide-bound hyaluronic acid hydrogel, the method for preparing a ceramide-bound hyaluronic acid hydrogel, and the composition including the ceramide-bound hyaluronic acid hydrogel have been described above, the detailed description thereof will be omitted.

In an embodiment, the method for preparing a composition may be a method for preparing a solubilized composition, but is not limited thereto. For the method for preparing a composition according to one aspect of the present disclosure, it is possible to prepare a composition including both a ceramide and hyaluronic acid, which have different physical properties, without going through an emulsification process using a hyaluronic acid to which a ceramide or a pseudoceramide is bound.

In an embodiment, an emulsification process may not be performed in the method for preparing a composition.

In an embodiment, the mixing step may be performed at room temperature, but is not limited thereto. Meanwhile, since an emulsification process that has been usually used in the related art in order to prepare a composition including a ceramide and hyaluronic acid is performed at high temperatures, the emulsification process is different from the method for preparing a composition according to one aspect of the present disclosure.

In an embodiment, the method for preparing a composition may further include adding other components that may typically be included in the composition to the purified water. Since other components that may be included in the composition have been described above, the detailed description thereof will be omitted.

In an embodiment, the adding of the other components to the purified water may be performed simultaneously with the mixing step. In an embodiment, the adding of the other components to the purified water may be performed after or before the mixing step.

Hereinafter, the present invention will be described in detail with reference to Examples for specifically describing the present invention. The following Examples are provided for illustrative purposes only to aid in the understanding of the present invention, and the scope and spirit of the present invention are not limited thereby.

### Examples—Preparation of Ceramide-Bound Hyaluronic Acid Hydrogel

### 1. Preparation of Ceramide Emulsified Composition

An emulsified composition (emulsified base) including a ceramide or a pseudoceramide was prepared according to the composition in the following Table 1. Specifically, after a pseudoceramide and/or a ceramide NP, cholesterol and an emulsifier were added to glycerin or water, the resulting mixture was heated to 80° C. and dissolved, and then homogenized at 6,000 rpm for 3 minutes to prepare a ceramide emulsified composition.

Preparation Examples 4 and 5 are glycerin-based oil bases (oil in polyol bases; O/P bases) prepared using glycerin instead of water, and when dispersed in water, nano-emulsified particles are produced by D-phase emulsification. Preparation Examples 1 and 2 are 28 wt % and 19 wt % of a pseudoceramide, respectively, and are ceramide emulsified bases prepared using water without glycerin, and Preparation Example 3 is a 10 wt % ceramide NP, and is a ceramide emulsified base prepared using water without glycerin.

TABLE 1

| | | Preparation | Preparation | Preparation | Preparation | Preparation |
|---|---|---|---|---|---|---|
| Configuration (unit: wt %) | | Example 1 | Example 2 | Example 3 | Example 4 | Example 5 |
| Ceramide or | Ceramide PC-104 | 28 | 19 | 0 | 19 | 28 |
| pseudoceramide | (pseudoceramide) | | | | | |
| | Ceramide NP | 1 | 1 | 10 | 1 | 1 |
| Cholesterol | | 1 | 1 | 5 | 1 | 1 |
| Emulsifier | Polyglyceryl- | 6 | 5 | 6 | 6 | 6 |
| | 10 stearate | | | | | |
| | Cetyl phosphate | 0.2 | 0.2 | 0.7 | 0.7 | 0 |

| Glycerin | 0 | 0 | 0 | 72.3 | 54.7 |
|---|---|---|---|---|---|
| Water (D.I) | Residual | Residual | Residual | 0 | 0 |
| | amount | amount | amount | | |
| | (To 100) | (To 100) | (To 100) | | |

The size and polydispersity index of the emulsified particles of the emulsified compositions in Preparation Examples 1 to 5 were measured. The size and polydispersity index of the emulsified particles were measured using a Zeta-sizer (Zetasizer Nano ZS, Malvern). The size of the emulsified particles in Preparation Examples 1 to 3 prepared using water was within a range of 150 nm to 300 nm. When hyaluronic acid hydrogels were prepared using the emulsified bases in Preparation Examples 4 and 5, no gel was prepared. Since there was a concern that glycerin could inhibit the reaction between hyaluronic acid and the cross-linking agent, the emulsified bases in Preparation Examples 4 and 5 were excluded from subsequent experiments.

TABLE 2

| | Preparation | Preparation | Preparation | Preparation | Preparation |
|---|---|---|---|---|---|
| Classification | Example 1 | Example 2 | Example 3 | Example 4 | Example 5 |
| Emulsified particle | 233.4 | 232.3 | 298 | 159.6 | 268 |
| size | | | | | |
| Polydispersity index | 0.302 | 0.26 | 0.238 | 0.334 | 0.26 |
| (PDI) | | | | | |

### 2. Preparation of Ceramide-Bound Hyaluronic Acid Hydrogel

Ceramide-bound hyaluronic acid hydrogels were prepared according to the compositions shown in the following Table 3. In Table 3, PEGDE is polyethylene glycol diglycidyl ether, STMP is sodium trimetaphosphate, and hyaluronic acid is sodium hyaluronate (molecular weight Mw about 1 million Da).

Specifically, after the ceramide emulsified composition in Preparation Example 2 in Table 1 was dispersed in a 0.24 N KOH solution using a homomixer, hyaluronic acid and a cross-linking agent (PEGDE or STMP) were added, and then the resulting mixture was uniformly mixed using a mixer. The mixture was reacted in a 37° C. incubator for 24 hours, the resulting gel mixture was neutralized to a pH of about 7 by adding acetic acid, and then the neutralized gel was placed in a washing net and washed and swollen with purified water for 24 hours. Thereafter, the swollen gel was pulverized to a size of 50 μm using an extruder equipped with a 100 μm sieve to prepare a hydrogel.

TABLE 3

| | | Comparative | Comparative | Example | Example | Example |
|---|---|---|---|---|---|---|
| Classification (units: wt %) | | Example 1 | Example 2 | 1 | 2 | 3 |
| Ceramide emulsified | | 10 | 20 | 20 | 20 | 20 |
| composition | | | | | | |
| (Preparation Example 2) | | | | | | |
| KOH (0.24N) | | 77.6 | 47.6 | 57.6 | 50 | 45 |
| Hyaluronic acid | | 10 | 30 | 20 | 20 | 20 |
| Cross- | PEGDE | 2.4 | 2.4 | 2.4 | 0 | 0 |
| linking | STMP | 0 | 0 | 0 | 10 | 15 |
| agent | | | | | | |

In Comparative Example 1, in which hyaluronic acid was mixed in an amount of 10 wt % based on a total weight, the hydrogel was not cross-linked, and in Comparative Example 2, in which hyaluronic acid was mixed in an amount of 30 wt % based on the total weight, no hydrogel was prepared because the mixture was not mixed due to the high viscosity problem.

### Experimental Examples—Physical Properties and Efficacy of Ceramide-Bound Hyaluronic Acid Hydrogel

### 1. Evaluation of Physical Properties of Ceramide-Bound Hyaluronic Acid Hydrogel

The viscoelasticity of the hydrogels in Examples 1 to 3 prepared in '2. Preparation of ceramide-bound hyaluronic acid hydrogel' above was measured using a rheometer (KINEXUS Pro+, USA). Specifically, elasticity (storage modulus, G′) and viscosity (loss modulus, G″) were measured at 25° C. using a 20 mm plate under the conditions of a frequency range of 0.1 to 10 Hz, 1 Pa shear stress, and 1 mm gap. The values of G′, G″, and Tan δ at 1 Hz are shown in Table 4, and the value of Tan δ was calculated using the following equation: Tan δ=G“/G′.

TABLE 4

| Classification | | | |
|---|---|---|---|
| (viscoelasticity at 1 Hz) | Example 1 | Example 2 | Example 3 |
| Storage modulus (G′, Pa) | 983 | 112.4 | 756.2 |
| Loss modulus (G″, Pa) | 84.99 | 121.2 | 274.8 |
| Tanδ | 0.086 | 1.078 | 0.363 |

As disclosed in ‘2. Preparation of ceramide-bound hyaluronic acid hydrogel’, in Comparative Examples 1 and 2, hydrogels were not prepared, and therefore, viscoelasticity could not be measured. It was confirmed that the hydrogel in Example 1, in which PEGDE was used as a cross-linking agent, was relatively hard with a storage modulus of 983 Pa, a loss modulus of 84.99 Pa, and a Tand value of 0.086, and the hydrogel in Example 2, in which STMP was used as a cross-linking agent, had a slightly low storage modulus, a high loss modulus, and a high viscosity with a Tand value of 1.078. In Example 3, in which the STMP content was increased, the storage modulus and loss modulus increased. By adjusting the type and content of cross-linking agent, it is possible to prepare hyaluronic acid-ceramide hydrogels with various physical properties.

### 2. Evaluation of Degree of Swelling and Ceramide Content of Ceramide-Bound Hyaluronic Acid Hydrogel

The degree of swelling and ceramide content of the example hydrogel prepared in ‘2. Preparation of ceramide-bound hyaluronic acid hydrogel’ were analyzed.

The degree of swelling was evaluated by numerically calculating the water content of the hydrogel. Specifically, after the weight of the prepared hydrogel was weighed, the hydrogel was completely dried in an oven at 60° C. for 12 hours to measure the weight, and then the weight before drying relative to the weight after drying (weight before drying/weight after drying) was calculated. As shown in the following Table 5, it was confirmed that the degree of swelling varies depending on the cross-linking conditions. The theoretical value of the degree of swelling was derived based on the fact that when the degree of swelling is 100-fold, the hyaluronic acid content is 1% and the ceramide content is 0.2%.

In addition, it was confirmed that when the ceramide-bound hyaluronic acid hydrogel was prepared, ceramide emulsified particles were removed during the washing process after cross-linking, and the like. Ceramide content analysis was performed by HPLC, 1 g of a hydrogel was diluted with 10 ml of methanol and filtered through a 0.45 μm filter, and then HPLC analysis was performed under the following conditions:

### HPLC Analysis Conditions

Column: C18 column 5 μm, 250 mm×4.6 mm
Mobile phase: Acetonitrile: Methanol=7:3
Temperature: 20° C.
Flow rate: 0.8 mL/min
UV Detector: 210 nm
Waters HPLC.

As a result of comparing the theoretical value of the ceramide content according to the degree of swelling with the measured value in Table 5, it was confirmed that the hydrogel according to an embodiment of the present disclosure included ceramide in an amount of up to about 0.25 wt % based on the total weight, and the result is within the measurement error range, indicating that there is no loss of ceramide in the preparation process.

TABLE 5

Ceramide content (%)

| | Degree of | Theoretical | Measured |
|---|---|---|---|
| Classification | swelling | value | value |
| Example 1 | 110-fold | 0.181 | 0.196 |
| Example 2 | 90-fold | 0.22 | 0.23 |

### 3. Image Analysis of Ceramide-Bound Hyaluronic Acid Hydrogel

The images of the example hydrogel prepared in '2. Preparation of ceramide-bound hyaluronic acid hydrogel' were analyzed.

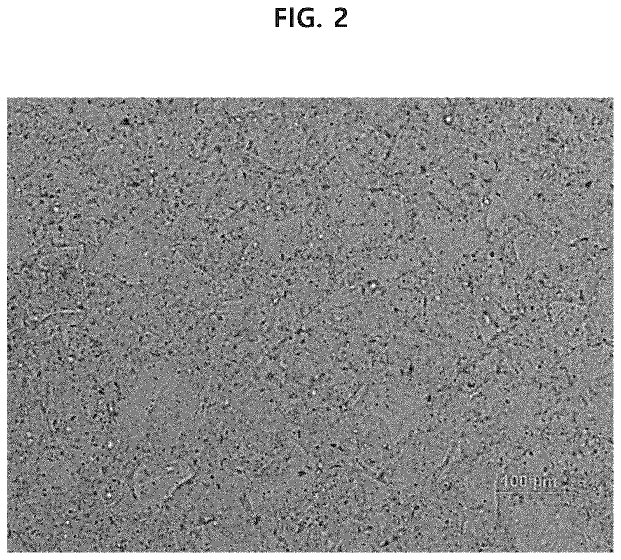

Specifically, the hydrogel in Example 3 prepared in '2. Preparation of ceramide-bound hyaluronic acid hydrogel' was observed at a magnification of 100× using a microscope (Nikon eclipse). As a result, it was confirmed that emulsified particles with a size of about 1 μm to 5 μm were included in the hydrogel (see FIG. 2).

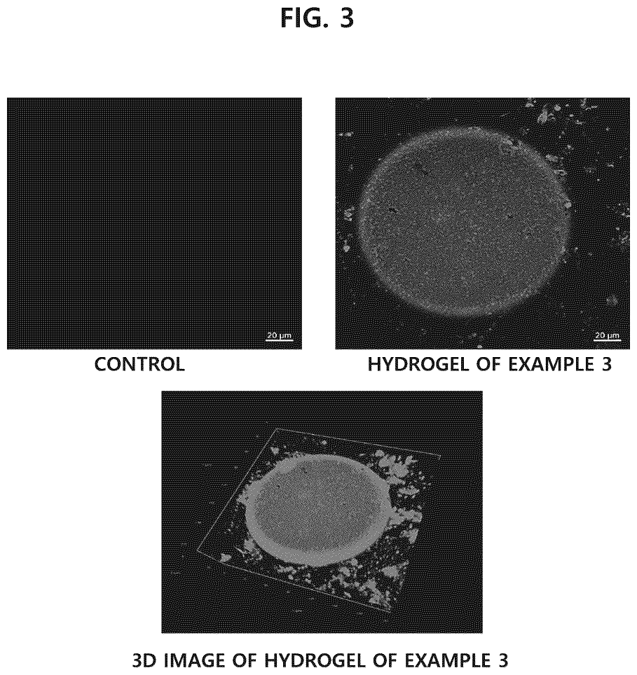

To distinguish the ceramide more precisely, the hydrogel in Example 3 was additionally observed using a confocal microscope (Carl Zeiss LSM980 NLO). The hydrogel of the example was diluted 1:100 with Nile-red O solution and then stained at room temperature for 1 hour. The stained hydrogel was placed on the confocal microscope stage using a pipette and then observed at a magnification of ×400. As a result, the control hydrogel without ceramide appeared black, meaning that it was not stained with Nile-red O. Meanwhile, the hydrogel in Example 3 showed an overall red color (shown in gray in FIG. 3). Through this, it could be confirmed that the ceramide was stained and distributed throughout the gel. The control in FIG. 3 is a hyaluronic acid hydrogel that does not include ceramide emulsified particles, and the types of basic solution, hyaluronic acid, and cross-linking agent used in preparing the hydrogel are the same as those in Example 3.

### 4. Evaluation of Composition Including Ceramide-Bound Hyaluronic Acid Hydrogel

A composition including the hydrogel of the example prepared in '2. Preparation of ceramide-bound hyaluronic acid hydrogel' was prepared, and its physical properties and efficacy were evaluated.

### (1) Preparation of Composition Including Ceramide and Hyaluronic Acid

A composition (ampule) including a ceramide and hyaluronic acid was prepared according to the composition in the following Table 6.

Example 4 in Table 6 is a composition including the hydrogel in Example 3 prepared in ‘2. Preparation of ceramide-bound hyaluronic acid hydrogel.’ Specifically, all the components shown in the following Table 6 were introduced at room temperature and then mixed at about 3,000 rpm for 3 minutes to prepare the composition in Example 4.

Comparative Example 3 in Table 6 is an emulsified composition in which a ceramide and a pseudoceramide are emulsified. In Comparative Example 3, the ceramide and the pseudoceramide were emulsified in the same content as in the hydrogel of Example 3 prepared in ‘2. Preparation of ceramide-bound hyaluronic acid hydrogel.’ Specifically, the ceramide and the pseudoceramide, cholesterol and an emulsifier were warmed to 80° C., and then added to water warmed to 70° C., and the resulting mixture was emulsified at about 6,000 rpm for 3 minutes. The obtained emulsified composition was cooled to about 50° C., and the remaining components were added thereto to prepare a composition of Comparative Example 3.

TABLE 6

| | | Comparative | |
|---|---|---|---|
| Classification (units: wt %) | | Example 3 | Example 4 |
| Ceramide-bound hyaluronic acid hydrogel | | 0 | 10 |
| (hydrogel of Example 3) | | | |
| Ceramide or | Ceramide PC-104 (pseudoceramide) | 0.019 | 0 |
| pseudoceramide | Ceramide NP | 0.001 | 0 |
| Cholesterol | | 0.001 | 0 |
| Emulsifier | Polyglyceryl-10 stearate | 0.05 | 0 |
| | Cetyl phosphate | 0.0002 | 0 |
| Purified water | | Residual | Residual |
| | | amount | amount |
| | | (to 100) | (to 100) |
| Glycerin | | 8 | 8 |
| 1,3-Butylene glycol (1,3 BG) | | 8 | 8 |
| Carbopol 981 | | 0.15 | 0.15 |
| Sodium hyaluronate | | 0.1 | 0 |
| Tromethamine | | 0.08 | 0.08 |
| Preservative | 1,2 hexanediol | 1.5 | 1.5 |
| | Ethylhexylglycerin | 0.05 | 0.05 |

To prepare a composition including both a ceramide and hyaluronic acid, a process is required in which the ceramide must be emulsified at high temperatures, as in the preparation method of Comparative Example 3 described above. And, it is difficult to prepare the composition as a composition of a solubilized formulation which has not been subjected to an emulsification process. Meanwhile, the ceramide-bound hyaluronic acid hydrogel according to one aspect of the present disclosure may be dispersed in water (or purified water) for formulation at room temperature as in the preparation method of Example 4 described above, and can also be prepared as a composition of a solubilized formulation. The contents of hyaluronic acid and ceramide included in the composition can be adjusted by varying the content of the gel.

### (2) Evaluation of Appearance of Composition

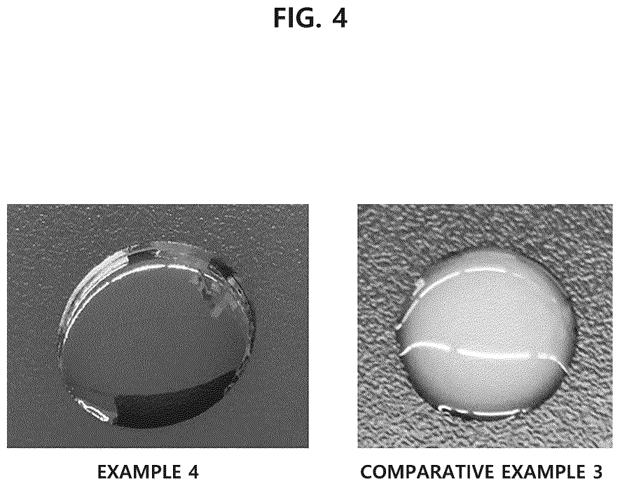

The composition of Example 4 (ampule including 10 wt % of the ceramide-bound hyaluronic acid hydrogel) ‘4. (1) Preparation of composition including ceramide and hyaluronic acid’ above was relatively transparent. Meanwhile, the composition of Comparative Example 3 in which the same contents of hyaluronic acid and the ceramide were emulsified was a white suspension formulation, which can be seen as a typical form of an emulsified formulation (FIG. 4).

### (3) Evaluation of Moisturizing Power of Composition

The moisturizing powers of the compositions of the Examples and Comparative Examples prepared in '4. (1) Preparation of composition including ceramide and hyaluronic acid' above were compared.

Skin moisture content was measured at an experimental site (2×2 cm area) by a skin stratum corneum hydration (SCH) sensor using a skin moisture meter (GP Skin, G Power). Moisture measurement results may range up to 100%, and generally, a measurement value 60% or less is displayed as dry, 80% or less as normal, and 80% or more as sufficient (moisture). First, the forearm of a subject was washed with a detergent to remove any remaining moisturizing components on the skin, and left to stand for 10 minutes, and then the skin moisture content at the experimental site was measured (control). 20 μl of ampules of Example 4 and Comparative Example 3 were applied to the experimental sites and allowed to be absorbed, and the changes in the skin moisture content were measured after 10 minutes, 30 minutes, 1 hour, and 3 hours.

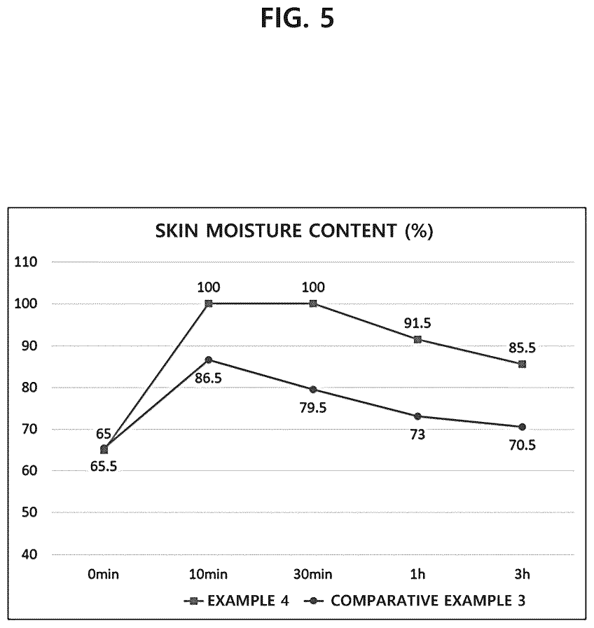

As a result of the measurement, the initial skin moisture content (before treatment in the Examples and Comparative Examples) was about 65%. After treatment with the composition of Example 4 including a ceramide-bound hyaluronic acid hydrogel, the skin moisture content was measured to be 100% after 10 minutes, 100% after 30 minutes, 91.5% after 1 hour, and 85.5% after 3 hours. Meanwhile, upon treatment with the composition of Comparative Example 3, in which a ceramide and hyaluronic acid were emulsified, the skin moisture content was measured to be 86.5% after 10 minutes, 79.5% after 30 minutes, 73% after 1 hour, and 70.5% after 3 hours (FIG. 5). As a result, the composition of Example 4 including a ceramide-bound hyaluronic acid hydrogel had better moisturizing power than the composition of Comparative Example 3 in which the same content of ceramide was emulsified with hyaluronic acid.

### (4) Evaluation of Usability of Composition

The usabilities of the compositions of the Examples and Comparative Examples prepared in ‘4. (1) Preparation of composition including ceramide and hyaluronic acid’ above were compared by sensory evaluation.

The compositions of Example 4 and Comparative Example 3 were applied to the back of the hand of a specialist sensory panel, and the characteristics of adhesion, coolness, oiliness and slipperiness were evaluated by converting them into scores.

Specifically, seven trained sensory panelists in the skin care category evaluated the characteristics on a 20-point scale (0 (not felt) to 19 (felt very strongly)) using a sensory evaluation method (SOP) set for ampule/essence types.

As a result of the sensory evaluation, the composition of Example 4 including the ceramide-bound hyaluronic acid hydrogel had a strong adhesion and coolness, but a relatively low oiliness and slipperiness. Meanwhile, the composition of Comparative Example 3 in which the same content of ceramide was emulsified with hyaluronic acid had a relatively low adhesion and coolness, and in particular, slipperiness, which is unique to hyaluronic acid, was very strong (Table 7). It is determined that in the ceramide-bound hyaluronic acid hydrogel according to one aspect of the present disclosure, the slipperiness characteristics unique to hyaluronic acid disappear as the hyaluronic acid is cross-linked and the molecular weight increases.

TABLE 7

| Classification | Example 4 | Comparative Example 3 |
|---|---|---|
| Adhesion | 14 | 2 |
| Coolness | 18 | 12 |
| Oiliness | 2 | 8 |
| Slipperiness | 4 | 18 |

In summary, in the ceramide-bound hyaluronic acid hydrogel according to one aspect of the present disclosure, ceramide may be distributed in the hydrogel and exist stably. Furthermore, the ceramide-bound hyaluronic acid hydrogel according to one aspect of the present disclosure may be simply mixed with water (or purified water) at room temperature to prepare a composition (for example: a solubilized formulation), and the prepared composition has excellent moisturizing power and usability.

Meanwhile, in order to apply a ceramide to a composition together with hyaluronic acid without binding it to a hydrogel, there is a disadvantageous in that the process is not simple because an emulsification process performed at high temperatures is required. The composition prepared by the emulsification process as described above exhibits a white color suspended as an emulsified formulation, and thus has a relatively poor appearance. Furthermore, the composition prepared by the emulsification process does not have excellent moisturizing power or usability, even though the composition includes the same content of ceramide as the composition including the ceramide-bound hyaluronic acid hydrogel according to one aspect of the present disclosure.

The present disclosure is further illustrated by the following embodiments, which do not limit the scope of the claims.

Embodiment 1. A ceramide-bound hyaluronic acid hydrogel including: a hyaluronic acid hydrogel including hyaluronic acid or a salt thereof; and emulsified particles bound to the hyaluronic acid hydrogel;

wherein the emulsified particles include at least one of a ceramide or a pseudoceramide. Embodiment 2. The ceramide-bound hyaluronic acid hydrogel of Embodiment 1, wherein the ceramide-bound hyaluronic acid hydrogel includes the hyaluronic acid or the salt thereof in an amount of 0.1 wt % to 10 wt % based on a total weight of the ceramide-bound hyaluronic acid hydrogel.

Embodiment 3. The ceramide-bound hyaluronic acid hydrogel of any one of Embodiments 1 and 2,
wherein the ceramide-bound hyaluronic acid hydrogel includes at least one of the ceramide or the pseudoceramide in an amount of 0.01 wt % to 10 wt % based on a total weight of the ceramide-bound hyaluronic acid hydrogel.

Embodiment 4. The ceramide-bound hyaluronic acid hydrogel of any one of Embodiments 1 to 3,
wherein the hyaluronic acid hydrogel including the hyaluronic acid or the salt thereof is one in which hyaluronic acid or a salt thereof is chemically cross-linked by a cross-linking agent.

Embodiment 5. The ceramide-bound hyaluronic acid hydrogel of Embodiment 4,
wherein the cross-linking agent is at least one selected from the group consisting of a sulfone-based cross-linking agent, an epoxide-based cross-linking agent, and a phosphate-based cross-linking agent.

Embodiment 6. The ceramide-bound hyaluronic acid hydrogel of any one of Embodiments 4 to 5,
wherein the cross-linking agent is at least one selected from the group consisting of polyethylene glycol diglycidyl ether, sodium trimetaphosphate, epichlorohydrin, epibromohydrin, 1,4-butandiol diglycidyl ether, ethylene glycol diglycidyl ether, 1,6-hexanediol diglycidyl ether, propylene glycol diglycidyl ether, polypropylene glycol diglycidyl ether, polytetramethylene glycol diglycidyl ether, neopentyl glycol diglycidyl ether, polyglycerol polyglycidyl ether, diglycerol polyglycidyl ether, glycerol polyglycidyl ether, trimethylpropane polyglycidyl ether, 1,2-(bis(2,3-epoxypropoxy)ethylene), pentaerythritol polyglycidyl ether, and sorbitol polyglycidyl ether.

Embodiment 7. The ceramide-bound hyaluronic acid hydrogel of any one of Embodiments 1 to 6,
wherein the ceramide is at least one selected from the group consisting of ceramide NP, ceramide NS, ceramide AS, ceramide EOS, ceramide NDS, ceramide ADS, ceramide EODS, ceramide AP, ceramide EOP, ceramide NH, ceramide AH, ceramide EOH, and hydroxypalmitoyl sphinganine.

Embodiment 8. The ceramide-bound hyaluronic acid hydrogel of any one of Embodiments 1 to 7,
wherein the pseudoceramide is at least one selected from the group consisting of hydroxypropyl bispalmitamide MEA, hydroxypropyl bisstearamide MEA, hydroxypropyl bislauramide MEA, hydroxypropyl bis-isostearamide MEA, bis-hydroxyethyl tocopherylsuccinoylamido hydroxypropane, cetyl-PG hydroxyethyl palmitamide, myristoyl/palmitoyl oxostearamide/arachamide MEA, and bis-palmitoyl tromethamine.

Embodiment 9. The ceramide-bound hyaluronic acid hydrogel of any one of Embodiments 1 to 8,
wherein the emulsified particles have an average diameter of 0.1 μm to 100 μm.

Embodiment 10. The ceramide-bound hyaluronic acid hydrogel of any one of Embodiments 1 to 9,
wherein the ceramide-bound hyaluronic acid hydrogel has a Tan δ of 0.01 to 2, and the Tan δ is calculated by the following Mathematical Formula 1:

### Mathematical Formula 1

Tanδ=loss modulus measured at 25° C. (Pa)/storage modulus measured at 25° C. (Pa).

Embodiment 11. The ceramide-bound hyaluronic acid hydrogel of any one of Embodiments 1 to 10,
wherein the ceramide-bound hyaluronic acid hydrogel has a degree of swelling of 10 to 300, and
the degree of swelling is measured by the following method for measuring a degree of swelling:

### Method for Measuring Degree of Swelling

Degree of swelling=weight of ceramide-bound hyaluronic acid hydrogel before drying/weight of ceramide-bound hyaluronic acid hydrogel measured after drying at 60° C. for 12 hours.

Embodiment 12. A composition including the ceramide-bound hyaluronic acid hydrogel of any one of Embodiments 1 to 11.

Embodiment 13. The composition of Embodiment 12, wherein the composition is a solubilized formulation or a transparent formulation.

Embodiment 14. A method for preparing the ceramide-bound hyaluronic acid hydrogel of any one of Embodiments 1 to 11, the method including:
(a) mixing at least one of a ceramide or a pseudoceramide, an emulsifier, and water to prepare a ceramide emulsified composition; and
(b) mixing the prepared ceramide emulsified composition with at least one of hyaluronic acid or a salt thereof, and a cross-linking agent to obtain a hyaluronic acid hydrogel to which emulsified particles including at least one of a ceramide or a pseudoceramide are bound.

Embodiment 15. The method of Embodiment 14, wherein the ceramide emulsified composition includes ceramide emulsified particles.

Embodiment 16. The ceramide-bound hyaluronic acid hydrogel of any one of Embodiments 14 to 15,
wherein step (b) is
mixing the ceramide emulsified composition prepared in step (a) with hyaluronic acid or a salt thereof, and a cross-linking agent to obtain a gel mixture, and allowing the obtained gel mixture to be swollen and then pulverized to obtain a hyaluronic acid hydrogel to which emulsified particles including at least one of a ceramide or a pseudoceramide are bound.

Embodiment 17. A method for preparing a composition, the method including: mixing the ceramide-bound hyaluronic acid hydrogel of any one of Embodiments 1 to 11 with purified water.

Embodiment 18. The method of Embodiment 17, wherein the mixing step is performed at room temperature.

Embodiment 19. A method for improving the skin, the method including: applying the composition of any one of Embodiments 12 and 13 to a subject.

Embodiment 20. The method of Embodiment 19, wherein the improvement of the skin enhances skin moisture or strengthens the skin barrier.

What is claimed is:

1. A ceramide-bound hyaluronic acid hydrogel comprising:
a hyaluronic acid hydrogel comprising hyaluronic acid or a salt thereof; and
emulsified particles bound to the hyaluronic acid hydrogel;
wherein the emulsified particles comprise at least one of a ceramide or a pseudoceramide.

2. The ceramide-bound hyaluronic acid hydrogel of claim 1, wherein the ceramide-bound hyaluronic acid hydrogel comprises the hyaluronic acid or the salt thereof in an amount of 0.1 wt % to 10 wt % based on a total weight of the ceramide-bound hyaluronic acid hydrogel.

3. The ceramide-bound hyaluronic acid hydrogel of claim 1, wherein the ceramide-bound hyaluronic acid hydrogel comprises at least one of the ceramide or the pseudoceramide in an amount of 0.01 wt % to 10 wt % based on a total weight of the ceramide-bound hyaluronic acid hydrogel.

4. The ceramide-bound hyaluronic acid hydrogel of claim 1, wherein the hyaluronic acid hydrogel comprising the hyaluronic acid or the salt thereof is one in which hyaluronic acid or a salt thereof is chemically cross-linked by a cross-linking agent.

5. The ceramide-bound hyaluronic acid hydrogel of claim 4, wherein the cross-linking agent is at least one selected from the group consisting of a sulfone-based cross-linking agent, an epoxide-based cross-linking agent, and a phosphate-based cross-linking agent.

6. The ceramide-bound hyaluronic acid hydrogel of claim 4, wherein the cross-linking agent is at least one selected from the group consisting of polyethylene glycol diglycidyl ether, sodium trimetaphosphate, epichlorohydrin, epibromohydrin, 1,4-butandiol diglycidyl ether, ethylene glycol diglycidyl ether, 1,6-hexanediol diglycidyl ether, propylene glycol diglycidyl ether, polypropylene glycol diglycidyl ether, polytetramethylene glycol diglycidyl ether, neopentyl glycol diglycidyl ether, polyglycerol polyglycidyl ether, diglycerol polyglycidyl ether, glycerol polyglycidyl ether, trimethylpropane polyglycidyl ether, 1,2-(bis(2,3-epoxypropoxy) ethylene), pentaerythritol polyglycidyl ether, and sorbitol polyglycidyl ether.

7. The ceramide-bound hyaluronic acid hydrogel of claim 1, wherein the ceramide is at least one selected from the group consisting of ceramide NP, ceramide NS, ceramide AS, ceramide EOS, ceramide NDS, ceramide ADS, ceramide EODS, ceramide AP, ceramide EOP, ceramide NH, ceramide AH, ceramide EOH, and hydroxypalmitoyl sphinganine.

8. The ceramide-bound hyaluronic acid hydrogel of claim 1, wherein the pseudoceramide is at least one selected from the group consisting of hydroxypropyl bispalmitamide MEA, hydroxypropyl bisstearamide MEA, hydroxypropyl bislauramide MEA, hydroxypropyl bis-isostearamide MEA, bis-hydroxyethyl tocopherylsuccinoylamido hydroxypropane, cetyl-PG hydroxyethyl palmitamide, myristoyl/palmitoyl oxostearamide/arachamide MEA, and bis-palmitoyl tromethamine.

9. The ceramide-bound hyaluronic acid hydrogel of claim 1, wherein the emulsified particles have an average diameter of 0.1 μm to 100 μm.

10. The ceramide-bound hyaluronic acid hydrogel of claim 1, wherein the ceramide-bound hyaluronic acid hydrogel has a Tanδ of 0.01 to 2, and
the Tan δ is calculated by the following Mathematical Formula 1:
[Mathematical Formula 1]
Tan δ=loss modulus measured at 25° C. (Pa)/storage modulus measured at 25° C. (Pa).

11. The ceramide-bound hyaluronic acid hydrogel of claim 1, wherein the ceramide-bound hyaluronic acid hydrogel has a degree of swelling of 10 to 300, and
the degree of swelling is measured by the following method for measuring a degree of swelling:
[Method for Measuring Degree of Swelling]
Degree of swelling=weight of ceramide-bound hyaluronic acid hydrogel before drying/weight of ceramide-bound hyaluronic acid hydrogel measured after drying at 60° C. for 12 hours.

12. A composition comprising the ceramide-bound hyaluronic acid hydrogel of claim 1.

13. The composition of claim 12, wherein the composition is a solubilized formulation or a transparent formulation.

14. A method for preparing the ceramide-bound hyaluronic acid hydrogel of claim 1, the method comprising:
(a) mixing at least one of a ceramide or a pseudoceramide, an emulsifier, and water to prepare a ceramide emulsified composition; and
(b) mixing the prepared ceramide emulsified composition with at least one of hyaluronic acid or a salt thereof, and a cross-linking agent to obtain a hyaluronic acid hydrogel to which emulsified particles including at least one of a ceramide or a pseudoceramide are bound.

15. The method of claim 14, wherein the ceramide emulsified composition comprises ceramide emulsified particles.

16. The method of claim 14, wherein step (b) is
mixing the ceramide emulsified composition prepared in step (a) with hyaluronic acid or a salt thereof, and a cross-linking agent to obtain a gel mixture, and allowing the obtained gel mixture to be swollen and then pulverized to obtain a hyaluronic acid hydrogel to which emulsified particles including at least one of a ceramide or a pseudoceramide are bound.

17. A method for preparing a composition, the method comprising: mixing the ceramide-conjugated hyaluronic acid hydrogel of claim 1 with purified water.

18. The method of claim 17, wherein the mixing step is performed at room temperature.
